(19) United States
(12) Patent Application Publication
(10) Pub. No.: US 2018/0220926 A1
(43) Pub. Date: Aug. 9, 2018

(54) ELECTROMAGNETIC NAVIGATION SYSTEM WITH MAGNETO-RESISTIVE SENSORS AND APPLICATION-SPECIFIC INTEGRATED CIRCUITS

(71) Applicant: Boston Scientific Scimed Inc., Maple Grove, MN (US)

(72) Inventors: David W. Kelly, Eagan, MN (US); Matthew Hein, Eden Prairie, MN (US)

(21) Appl. No.: 15/888,418

(22) Filed: Feb. 5, 2018

Related U.S. Application Data
(60) Provisional application No. 62/455,316, filed on Feb. 6, 2017.

(51) Int. Cl. A61B 5/06; G01D 5/16

(52) U.S. Cl. CPC A61B 5/062; G01D 5/16

(57) ABSTRACT

A sensing apparatus includes a magneto-resistive (MR) sensing element for sensing magnetic fields and semiconductor circuitry including an output circuit coupled to the MR sensing element. The output circuit generates a sensing signal proportional to the sensed magnetic fields to be combined with a bias signal to the MR sensing element.

### CROSS-REFERENCE TO RELATED APPLICATION

This application claims priority to Provisional Application No. 62/455,316, filed Feb. 6, 2017, which is herein incorporated by reference in its entirety.

### TECHNICAL FIELD

The present disclosure relates to systems, methods, and devices for tracking items. More specifically, the disclosure relates to systems, methods, and devices for electro-magnetically tracking medical devices used in medical procedures.

### BACKGROUND

A variety of systems, methods, and devices can be used to track medical devices. Tracking systems can use externally generated magnetic fields that are sensed by at least one tracking sensor in the tracked medical device. The externally generated magnetic fields provide a fixed frame of reference, and the tracking sensor senses the magnetic fields to determine the location and orientation of the sensor in relation to the fixed frame of reference.

### SUMMARY

In Example 1, a sensing apparatus comprising a magneto-resistive (MR) sensing element for sensing magnetic fields, and semiconductor circuitry including an output circuit coupled to the MR sensing element. The output circuit generates a sensing signal proportional to the sensed magnetic fields to be combined with a bias signal to the MR sensing element, and the MR sensing element is formed on a first chip and the semiconductor circuitry is formed on a second chip.

In Example 2, the sensing apparatus of Example 1, further comprising a reset coil element for resetting a magnetic orientation of the MR sensing element, wherein the semiconductor circuitry further includes a reset configured to supply a reset current to the reset coil element.

In Example 3, the sensing apparatus of either of Examples 1 or 2, further comprising a tuning resistor, wherein the sensing signal is based on a resistance value of the tuning resistor.

In Example 4, the sensing apparatus of Example 3, wherein the reset coil element and the tuning resistor are formed on the first chip.

In Example 5, the sensing apparatus of any of Examples 1-4, wherein the first chip is placed in close proximity to the second chip.

In Example 6, the sensing apparatus of any of Examples 1-5, wherein the first chip and the second chip are electrically connected to one another.

In Example 7, the sensing apparatus of any of Examples 1-6, wherein the first chip is placed on top of the second chip.

In Example 8, a sensor assembly comprising a plurality of sensing apparatuses according to any of Examples 1-7 mechanically coupled to a substrate.

In Example 9, the sensor assembly of Example 8, wherein the substrate is a flexible substrate.

In Example 10, the sensor assembly of either of Examples 8 or 9, wherein the substrate includes a first portion oriented in a first plane and a second portion oriented in a second plane that is non-parallel to the first plane.

In Example 11, the sensor assembly of Example 10, wherein the second plane is oriented orthogonally to the first plane.

In Example 12, the sensor assembly of either of Examples 10 or 11, wherein a first one of the plurality of sensing apparatuses is supported by the first portion of the substrate, and a second one of the plurality of sensing apparatuses is supported by the second portion of the substrate.

In Example 13, a medical probe including a distal portion having a sensor assembly according to any of Examples 8-12.

In Example 14, a medical system comprising the medical probe according to Example 13, a magnetic field generator configured to generate a multi-dimensional magnetic field in a volume including the medical probe and a patient, and a processor operable to receive outputs from the sensor assembly to determine a position of the sensor assembly within the volume.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a schematic of a tracking system, in accordance with certain embodiments of the present disclosure.

FIG. 2 shows a schematic of sensor circuitry, in accordance with certain embodiments of the present disclosure.

FIG. 3 shows a schematic of sensor circuitry, in accordance with certain embodiments of the present disclosure.

FIG. 4 shows a schematic of sensor assembly circuitry, in accordance with certain embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the invention as defined by the appended claims.

### DETAILED DESCRIPTION

During medical procedures, medical devices such as probes (e.g., catheters) are inserted into a patient through the patient's vascular system and/or a catheter lumen. To track the location and orientation of a probe within the patient, probes can be provisioned with magnetic field sensors.

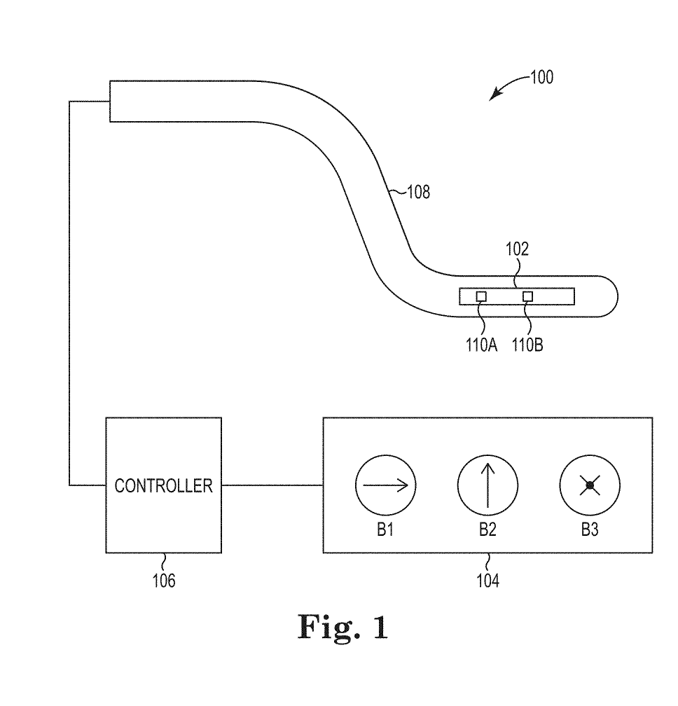

FIG. 1 is a diagram illustrating a tracking system 100 including a sensor assembly 102, a magnetic field generator 104, a controller 106, and a probe 108 (e.g., catheter, imaging probe, diagnostic probe). As shown, the sensor assembly 102 can be positioned within the probe 108, for example, at a distal end of the probe 108. The tracking system 100 is configured to determine the location and orientation of the sensor assembly 102 and, therefore, the probe 108. Magnetic fields generated by the magnetic field generator 104 provide a frame of reference for the tracking system 100 such that the location and orientation of the sensor assembly 102 within the generated magnetic field can be determined. The tracking system 100 can be used in a medical procedure, where the probe 108 is inserted into a patient and the sensor assembly 102 is used to assist with tracking the location of the probe 108 in the patient.

In various embodiments, the probe 108 may include, for example, a catheter (e.g., a mapping catheter, an ablation catheter, a diagnostic catheter, introducer, etc.), an endoscopic probe or cannula, an implantable medical device (e.g., a control device, a monitoring device, a pacemaker, an implantable cardioverter defibrillator (ICD), a cardiac resynchronization therapy (CRT) device, a CRT-D device, etc.), and/or the like. For example, in embodiments, the probe 108 may include a mapping catheter associated with an anatomical mapping system. The probe 108 may include any other type of device configured to be at least temporarily disposed within a subject.

The sensor assembly 102 is communicatively coupled to the controller 106 by a wired or wireless communications path such that the controller 106 sends and receives various signals to and from the sensor assembly 102. The magnetic field generator 104 is configured to generate one or more magnetic fields. For example, the magnetic field generator 104 is configured to generate at least three magnetic fields B1, B2, and B3, each generated by a respective magnetic field transmitter (e.g., a coil). The controller 106 is configured to control the magnetic field generator 104 via a wired or wireless communications path to generate one or more of the magnetic fields B1, B2, and B3 to assist with tracking the sensor assembly 102 (and therefore probe 108).

In various embodiments, the controller 106 includes a signal generator configured to provide driving current to each of the magnetic field transmitters, causing each magnetic field transmitter assembly to transmit an electromagnetic field. In certain embodiments, the controller 106 is configured to provide variable (e.g., sinusoidal) driving currents to the magnetic field transmitters within the magnetic field generator 104. The controller 106 can be implemented using firmware, integrated circuits, and/or software modules that interact with each other or are combined together. For example, the controller 106 may include computer-readable instructions/code for execution by a processor within or associated with the controller 106. Such instructions may be stored on a non-transitory computer-readable medium and transferred to the processor for execution. In some embodiments, the controller 106 can be implemented in one or more application-specific integrated circuits and/or other forms of circuitry suitable for controlling and processing magnetic tracking signals and information.

The sensor assembly 102 is configured to sense the generated magnetic fields and provide tracking signals indicating the location and orientation of the sensor assembly 102 in up to six degrees of freedom (i.e., x, y, and z measurements, and pitch, yaw, and roll angles). Generally, the number of degrees of freedom that a tracking system is able to track depends on the number of magnetic field sensors and magnetic field generators. For example, a tracking system with a single magnetic field sensor may not be capable of tracking roll angles and thus are limited to tracking in only five degrees of freedom (i.e., x, y, and z coordinates, and pitch and yaw angles). This is because a magnetic field sensed by a single magnetic field sensor does not change as the single magnetic field sensor is “rolled.” As such, the sensor assembly 102 includes at least two magnetic field sensors, 110A and 110B. The magnetic field sensors can include sensors such as inductive sensing coils and/or various sensing elements such as magneto-resistive (MR) sensing elements (e.g., anisotropic magneto-resistive (AMR) sensing elements, giant magneto-resistive (GMR) sensing elements, tunneling magneto-resistive (TMR) sensing elements, Hall effect sensing elements, colossal magneto-resistive (CMR) sensing elements, extraordinary magneto-resistive (EMR) sensing elements, spin Hall sensing elements, and the like), giant magneto-impedance (GMI) sensing elements, and/or flux-gate sensing elements. In addition, the sensor assembly 102 and/or the probe 108 can feature other types of sensors, such as temperature sensors, ultrasound sensors, etc.

The sensor assembly 102 is configured to sense each of the magnetic fields B1, B2, and B3 and provide signals to the controller 106 that correspond to each of the sensed magnetic fields B1, B2, and B3. The controller 106 receives the signals from the sensor assembly 102 via the communications path and determines the position and location of the sensor assembly 102 and probe 108 in relation to the generated magnetic fields B1, B2, and B3.

The magnetic field sensors can be powered by voltages or currents to drive or excite elements of the magnetic field sensors. The magnetic field sensor elements receive the voltage or current and, in response to one or more of the generated magnetic fields, the magnetic field sensor elements generate sensing signals, which are transmitted to the controller 106. The controller 106 is configured to control the amount of voltage or current to the magnetic field sensors and to control the magnetic field generators 104 to generate one or more of the magnetic fields B1, B2, and B3. The controller 106 is further configured to receive the sensing signals from the magnetic field sensors and to determine the location and orientation of the sensor assembly 102 (and therefore probe 108) in relation to the magnetic fields B1, B2, and B3. The controller 106 can be implemented using firmware, integrated circuits, and/or software modules that interact with each other or are combined together. For example, the controller 106 may include computer-readable instructions/code for execution by a processor. Such instructions may be stored on a non-transitory computer-readable medium and transferred to the processor for execution. In general, the controller 106 can be implemented in any form of circuitry suitable for controlling and processing magnetic tracking signals and information.

In the illustrated embodiment the controller 106 is shown as a single functional block that controls the operation of the magnetic field generator 104 and also receives and processes the signals from the sensor assembly 102 corresponding to the sensed magnetic fields B1, B2, B3 for tracking the position and orientation of the probe 108 within the multi-dimensional magnetic field generated by the magnetic field generator 104. The skilled artisan will appreciate that the foregoing functionality may be implemented in one or more hardware and software components/systems. For example, in embodiments, the controller 106 functionality relating to control of the magnetic field generator 104 and the processing of the signals from the sensor assembly 102 may be performed by a single processor. In other embodiments, these functions may be performed in multiple processors.

In various embodiments, the magnetic field sensors 110a, 110b are disposed on a substrate as part of the sensor assembly 102. In embodiments, the substrate may be a flexible substrate. In embodiments, the magnetic field sensors 110a, 110b may be oriented so as to be sensitive to components of the generated magnetic field in different directions. In embodiments, the directions of sensitivity may be orthogonal to one another. In various embodiments, the magnetic field sensors 110a, 110b may lie in the same plane, but be oriented in different directions. In other embodiments, the substrate may include a first portion oriented in a first plane, with the magnetic field sensor 110a being located thereon, and may also include a second portion oriented in a second plane with the magnetic field sensor 110b located thereon. In embodiments, the first and second planes may be orthogonal to one another.

Although in the illustrated embodiment the sensor assembly 102 includes two magnetic field sensors 110a, 110b, in other embodiments the sensor assembly 102 may include additional magnetic field sensors.

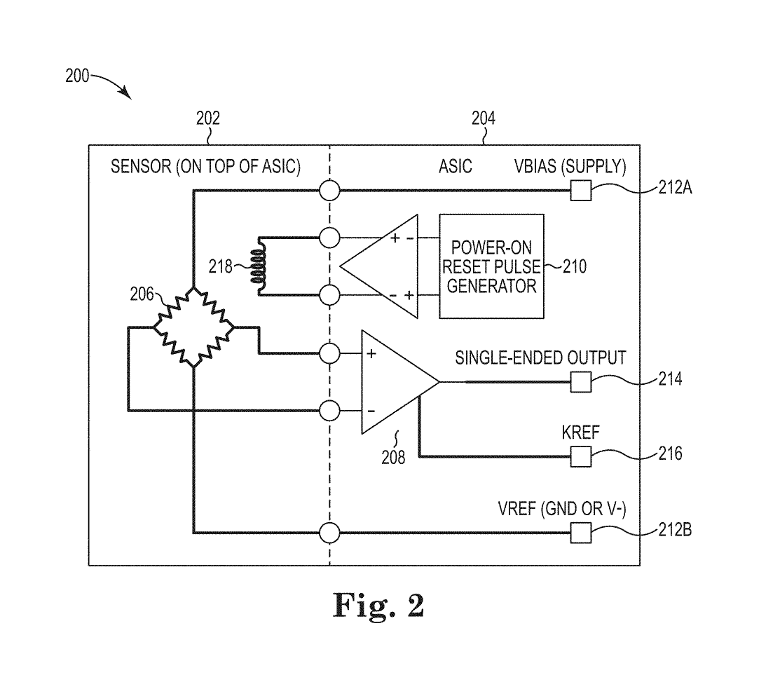

FIG. 2 shows sensor circuitry 200 for a magnetic field sensor such as the magnetic field sensor 110A or 110B of FIG. 1. The sensor circuitry 200 includes a sensor portion 202 and an application-specific integrated circuit (ASIC) portion 204. As shown in FIG. 2, the sensor portion 202 and the ASIC portion 204 can be implemented on the same die or substrate (e.g., a monolithic design). For example, the sensor portion 202 can be fabricated on top of the ASIC portion 204. In some embodiments, the sensor portion 202 and the ASIC portion 204 can be implemented on separate dies and positioned next to each other. In such embodiments, the sensor portion 202 and the ASIC portion 204 can be electrically and communicatively coupled together.

The sensor portion 202 includes one or more MR sensing elements 206, which can be AMR sensing elements, GMR sensing elements, TMR sensing elements, CMR sensing elements, EMR sensing elements, and the like. The MR sensing elements 206 are configured to sense magnetic fields, like those generated by the magnetic field generator 104 of FIG. 1, and generate a sensing signal. In some embodiments, the MR sensing elements 206 can be arranged in a Wheatstone bridge configuration as shown in FIG. 2, where four MR sensing elements are connected together to make a bridge circuit. In such embodiments, a change in one or more of the MR sensing elements in the bridge circuit, due to the sensed magnetic field, will result in a differential voltage output from the bridge circuit, so as to generate the sensing signal. In some embodiments, a single MR sensing element can be used to sense magnetic fields.

The ASIC portion 204 includes various integrated circuits such as an amplifier circuit 208 and a reset circuit 210, which can be fashioned using any suitable semiconductor technology. The ASIC portion 204 also includes bias connections, 212A and 212B, which are used to provide a bias current to the MR sensing elements 206 from a supply source (not shown), and also to provide power to the ASIC portion 204.

The amplifier circuit 208 operates to increase the signal strength of the generated responsive sensing signal from the MR sensing elements 206. Accordingly, the amplifier circuit 208 includes an output connection 214 and a Kelvin connection 216. The Kelvin connection 216 is operable to compensate for voltage losses caused by line resistances, which would otherwise cause errors in low voltage measurements, and to define the reference voltage for the amplifier circuit 208 output (i.e., when the input signal to the amplifier circuit 208 is zero, the output from the amplifier circuit 208 is equal to the reference voltage).

The reset circuit 210 operates to reset the one or more MR sensing elements 206. Accordingly, the reset circuit 210 includes a reset coil 218 constructed near the MR sensing elements 206 on the sensor portion 202. After exposure to external magnetic fields such as the magnetic fields B1, B2, and B3 of FIG. 1, the MR sensing elements 206 typically require the application of a magnetic field to reset their magnetic sensitivities. That is, by resetting the magneto-resistive film domains in the MR sensing elements 206 to a previous or relatively-known magnetic orientation. This is accomplished when the reset circuit 210 generates a current pulse through the reset coil 218 to create the magnetic field needed for the reset. For example, the reset circuit 210 can generate the current pulse at the system power-on stage to reset the MR sensing elements 206.

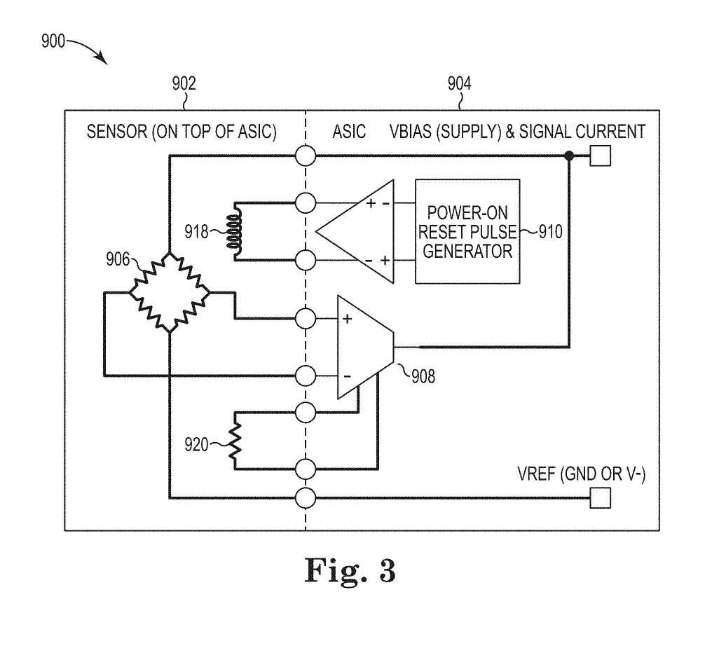

FIG. 3 shows sensor circuitry 900 for current mode signaling of a magnetic field sensor such as the magnetic field sensor 110A or 110B of FIG. 1. The sensor circuitry 900 is similar to the sensor circuitry 200 and includes a sensor portion 902 and an ASIC portion 904. The sensor portion 902 includes MR sensing elements 906, a reset coil 918, and a tuning resistor 920. The ASIC portion 904 includes various integrated circuits such as an output circuit 908 and a reset circuit 910 that controls the reset coil 918.

The sensor circuitry 900 sends the generated sensing signal from the MR sensing elements 906 back to the MR sensing elements 906 via the same signal line used to provide the bias current. A controller, such as the controller 106 of FIG. 1, would then separate the AC sensing signal from the DC bias. This approach has the advantage of minimizing the number of signals lines required. However, this approach also requires performing a power-on reset. The tuning resistor 920 can be used to set the gain of the output circuit 908. The value of tuning resistor 920 can be selected based on the MR sensing elements 906.

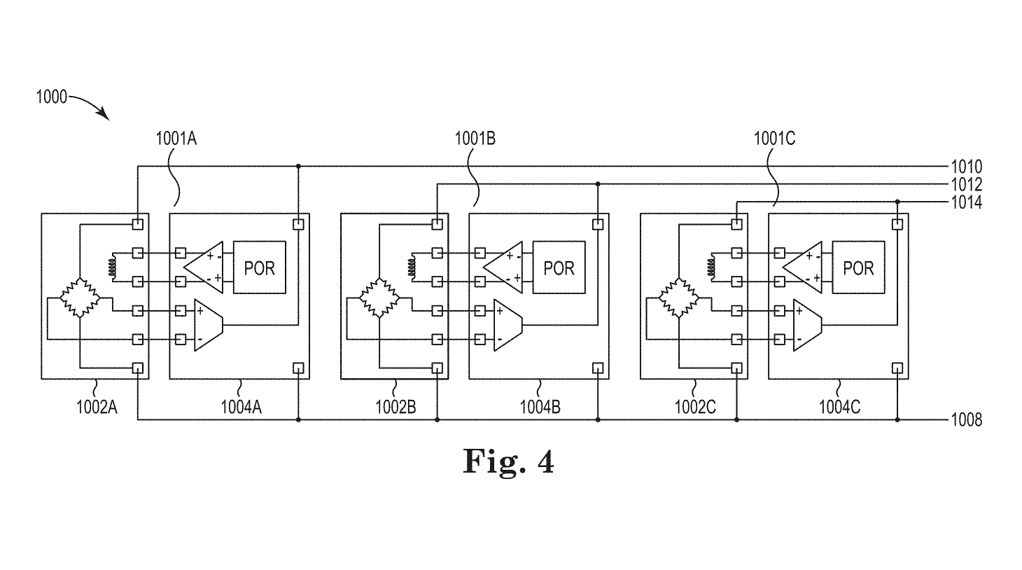

FIG. 4 shows sensor assembly circuitry 1000 for current mode signaling of a sensor assembly used in tracking systems such as the sensor assembly 102 used in the tracking system 100 of FIG. 1. The sensor assembly circuitry 1000 is comprised of a first sensor circuitry 1001A for a first magnetic field sensor, a second sensor circuitry 1001B for a second magnetic field sensor, and a third sensor circuitry 1001C for a third magnetic field sensor. The first sensor circuitry 1001A includes a separate sensor portion 1002A and a separate ASIC portion 1004A. Similarly, the second sensor circuitry 1001B includes a separate sensor portion 1002B and a separate ASIC portion 1004B, while the third sensor circuitry 1001C includes a separate sensor portion 1002C and a separate ASIC portion 1004C. However, each of the sensor circuitries 1001A-C can also be implemented monolithically like the sensor circuitry 900 of FIG. 3.

Similar to the sensor circuitry 900 of FIG. 3, each of the sensor circuitries 1001A-C combine the generated sensing signal from the magnetic field sensors with the bias signal. In this manner, the total number of signal lines is reduced as the bias signal line would replace the sensing signal line. As shown in FIG. 4, only four signal lines are needed: ground (1008) and three sensing signal/bias lines (1010-1014). Each magnetic field sensor would have its own dedicated sensing signal/bias line.

While FIG. 4 is shown as having three magnetic field sensors, it is appreciated that there could be only two magnetic field sensors or more than three magnetic field sensors. Moreover, while FIG. 4 shows that each magnetic field sensor is supported by an ASIC, in some embodiments, a single ASIC could support multiple magnetic field sensors.

The present disclosure provides the advantages of minimizing the number of signal lines when using MR technology. For example, the number of signal lines is reduced by using current mode signaling and by using the amplifier output for reset control. Moreover, by compensating for sensor bias current variations, the amount of magnetic coupling (crosstalk) from a distal sensor to a proximal sensor can be reduced.

It should be noted that, for simplicity and ease of understanding, the elements described above and shown in the figures are not drawn to scale and may omit certain features. As such, the drawings do not necessarily indicate the relative sizes of the elements or the non-existence of other features.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A sensing apparatus comprising:
a magneto-resistive (MR) sensing element for sensing magnetic fields; and
semiconductor circuitry including an output circuit coupled to the MR sensing element,
wherein the output circuit generates a sensing signal proportional to the sensed magnetic fields to be combined with a bias signal to the MR sensing element, and
wherein the MR sensing element is formed on a first chip and the semiconductor circuitry is formed on a second chip.

2. The sensing apparatus of claim 1, further comprising:
a reset coil element for resetting a magnetic orientation of the MR sensing element; and
wherein the semiconductor circuitry further includes a reset configured to supply a reset current to the reset coil element.

3. The sensing apparatus of claim 2, further comprising a tuning resistor, wherein the sensing signal is based on a resistance value of the tuning resistor.

4. The sensing apparatus of claim 3, wherein the reset coil element and the tuning resistor are formed on the first chip.

5. The sensing apparatus of claim 5, wherein the first chip and the second chip are electrically connected to one another.

6. The sensing apparatus of claim 5, wherein the first chip is located on top of the second chip.

7. A sensor assembly comprising a plurality of sensing apparatuses mechanically coupled to a substrate, wherein each of the plurality of sensing apparatuses comprises:
a magneto-resistive (MR) sensing element for sensing magnetic fields; and
semiconductor circuitry including an output circuit coupled to the MR sensing element,
wherein the output circuit generates a sensing signal proportional to the sensed magnetic fields to be combined with a bias signal to the MR sensing element, and
wherein the MR sensing element is formed on a first chip and the semiconductor circuitry is formed on a second chip.

8. The sensor assembly of claim 7, wherein the substrate is a flexible substrate.

9. The sensor assembly claim 8, wherein the substrate includes a first portion oriented in a first plane and a second portion oriented in a second plane that is non-parallel to the first plane.

10. The sensor assembly of claim 9, wherein the second plane is oriented orthogonally to the first plane.

11. The sensor assembly of claim 10, wherein a first one of the plurality of sensing apparatuses is supported by the first portion of the substrate, and a second one of the plurality of sensing apparatuses is supported by the second portion of the substrate.

12. The sensor assembly of claim 9, wherein each of the sensing apparatuses further comprises a tuning resistor, and the sensing signal for each sensing apparatus is based on a resistance value of the tuning resistor.

13. The sensor assembly of claim 12, wherein for each of the sensing apparatuses, the reset coil element and the tuning resistor are formed on the first chip.

14. The sensor assembly of claim 9, wherein for each of the sensing apparatuses, the first chip and the second chip are electrically connected to one another.

15. The sensor assembly of claim 14, wherein for each of the sensing apparatuses, the first chip is located on top of the second chip.

16. A medical probe comprising a distal portion having a sensor assembly disposed therein, wherein the sensor assembly includes a plurality of sensing apparatuses mechanically coupled to a substrate, wherein each of the plurality of sensing apparatuses comprises:
a magneto-resistive (MR) sensing element for sensing magnetic fields; and
semiconductor circuitry including an output circuit coupled to the MR sensing element,
wherein the output circuit generates a sensing signal proportional to the sensed magnetic fields to be combined with a bias signal to the MR sensing element, and
wherein the MR sensing element is formed on a first chip and the semiconductor circuitry is formed on a second chip.

17. The medical probe of claim 16, wherein each of the sensing apparatuses further comprises a tuning resistor, the sensing signal for each sensing apparatus is based on a resistance value of the tuning resistor.

18. The medical probe of claim 17, wherein for each of the sensing apparatuses, the reset coil element and the tuning resistor are formed on the first chip.

19. The medical probe of claim 16, wherein for each of the sensing apparatuses, the first chip and the second chip are electrically connected to one another.

20. The medical probe of claim 19, wherein for each of the sensing apparatuses, the first chip is located on top of the second chip.
